(19) United States
(12) Patent Application Publication
(10) Pub. No.: US 2009/0047381 A1
(43) Pub. Date: Feb. 19, 2009

(54) AMMONIA TRANSPORTER GENE AND USE THEREOF

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, OSAKA (JP)

(21) Appl. No.: 12/278,995

(22) PCT Filed: Dec. 5, 2006
(86) PCT No.: PCT/JP2006/324631

§ 371 Date: Aug. 11, 2008

(30) Foreign Application Priority Data
Feb. 24, 2006 (JP) 2006-049062

(51) Int. Cl. C12C 1/00; C07H 21/04; C07K 14/00; C12N 15/00; C12G 1/00; C12N 1/19; C12Q 1/68; C12Q 1/02

(52) U.S. Cl. 426/11; 536/23.1; 530/350; 435/320.1; 435/254.2; 435/6; 435/29; 426/62

(57) ABSTRACT

The present invention relates to ammonia transporter genes and use thereof. The invention relates in particular to a brewer's yeast which shows enhanced ammonia assimilation, alcoholic beverages produced using such yeast, and a method of producing such alcoholic beverages. More specifically, the invention relates to the MEP1 gene which codes for the ammonia transporter Mep1 in brewer's yeast, particularly to a yeast which can control the ammonia assimilation ability by controlling the level of expression of the nonScMEP1 gene or ScMEP1 gene characteristic to beer yeast and to a method of producing alcoholic beverages using such yeast.

### TECHNICAL FIELD

The present invention relates to ammonia transporter genes and use thereof. The invention relates in particular to a brewer's yeast which shows enhanced ammonia assimilation, alcoholic beverages produced using such yeast, and a method of producing such alcoholic beverages. More specifically, the invention relates to the MEP1 gene which codes for the ammonia transporter Mep1 in brewer's yeast, particularly to a yeast which can control the ammonia assimilation ability by controlling the level of expression of the nonScMEP1 gene or ScMEP1 gene characteristic to beer yeast and to a method of producing alcoholic beverages using such yeast.

### BACKGROUND ART

Ammonia and amino acids are known as nitrogen sources necessary for growth of yeasts. Also, during brewing, ammonia and amino acids contained in the raw material are assimilated as nitrogen sources for growth of the yeast.

In general, amino acids are important as taste components of alcoholic beverages and are known to be critical elements governing the quality of the products. Thus, it is important for developing a novel type of alcoholic beverage to control the amino acid content according to the quality of the alcoholic beverage of interest. For example, it is possible to add flavor and richness to the taste by increasing the amino acid content of an alcoholic beverage.

However, as noted above, amino acids as well as ammonia are assimilated by yeast as nitrogen sources during fermentation, and it is extremely difficult to control the amino acid content at the completion of fermentation.

To utilize extracellular amino acids and ammonia as nitrogen sources, the amino acids and ammonia must be transported into the yeast cells. It has been demonstrated that amino acids and ammonia transporters present in the yeast cell membrane are responsible for the transport of the amino acids and ammonia.

Three types of transporters having different substrate affinities (Mep1, Mep2 and Mep3) are known as the yeast ammonia transporters (Mol Cell Biol 17:4282-93, 1997). As the amino acid transporters, Gap1, with low substrate specificity, and a large numbers of other amino acid transporters having different substrate specificity are known, including the arginine transporter Can1 and the proline transporter Put4 (Curr Genet 36:317-28, 1999).

An example has hitherto been reported in which a yeast mutant having mutations in the genes involved in the transport of amino acids (gap1, shr3, can1, put4 and uga4) was used for controlling the amino acid content in alcoholic beverages (Japanese Examined Patent Publication (Kokai) No. 2001-321159).

### DISCLOSURE OF INVENTION

Under the above situations, there has been a need for yeast in which the assimilation of amino acid is regulated in order to control the amino acid content in the alcoholic beverages during its production. However, to control amount of amino acids remaining in the alcoholic beverages, it is necessary to control assimilation of nitrogen sources other than amino acids. Thus, it is desirable to provide a yeast in which assimilation of ammonia is controlled, whereby assimilation of amino acids is controlled.

The present inventors made extensive studies to solve the above problems and as a result, succeeded in identifying and isolating a gene encoding an ammonia transporter from beer yeast. Moreover, yeast which was transformed with the obtained gene for expression has been produced to verify the enhancement of the assimilation of ammonia, thereby completing the present invention.

Thus, the present invention relates to an ammonia transporter gene existing in a lager brewing yeast, to a protein encoded by said gene, to a transformed yeast in which the expression of said gene is controlled, to a method for producing alcoholic beverages using a yeast in which the expression of said gene is controlled, or the like. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO:1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 in which one or more amino acids thereof are deleted, substituted, inserted and/or added, and having ammonia transporter activity;

(d) a polynucleotide comprising a polynucleotide encoding a protein having an amino acid sequence having 60% or higher identity with the amino acid sequence of SEQ ID NO:2, and said protein having ammonia transporter activity;

(e) a polynucleotide comprising a polynucleotide which hybridizes to a polynucleotide consisting of a nucleotide sequence complementary to the nucleotide sequence of SEQ ID NO:1 under stringent conditions, and which encodes a protein having ammonia transporter activity; and

(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO:2 under stringent conditions, and which encodes a protein having ammonia transporter activity.

(2) The polynucleotide according to (1) above selected from the group consisting of:

(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has ammonia transporter activity;

(h) a polynucleotide comprising a polynucleotide encoding a protein having 90% or higher identity with the amino acid sequence of SEQ ID NO: 2, and having ammonia transporter activity; and

(i) a polynucleotide comprising a polynucleotide which hybridizes to a polynucleotide consisting of: a nucleotide sequence of SEQ ID NO: 1 or which hybridizes to a polynucleotide consisting of a nucleotide sequence complementary to the nucleotide sequence of SEQ ID NO: 1, under high stringent conditions, which encodes a protein having ammonia transporter activity.

(3) The polynucleotide according to (1) above comprising a polynucleotide consisting of the nucleotide sequence of SEQ ID NO: 1.

(4) The polynucleotide according to (1) above comprising a polynucleotide encoding a protein consisting of the amino acid sequence of SEQ ID NO: 2.

(5) The polynucleotide according to any one of (1) to (4) above, wherein the polynucleotide is DNA.

(6) A protein encoded by the polynucleotide according to any one of (1) to (5) above.

(7) A vector containing the polynucleotide according to any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(8) A vector containing any one of the polynucleotides selected from the group consisting of:

(j) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and having ammonia transporter activity;

(k) a polynucleotide comprising a polynucleotide encoding a protein having 90% or higher identity with the amino acid sequence of SEQ ID NO: 4, and having ammonia transporter activity; and

(l) a polynucleotide comprising a polynucleotide which hybridizes to the polynucleotide consisting of a nucleotide sequence of SEQ ID. NO: 3 or which hybridizes to a polynucleotide consisting of a nucleotide sequence complementary to the nucleotide sequence of SEQ ID NO: 3, under high stringent conditions, which encodes a protein having ammonia transporter activity.

(9) A polynucleotide selected from the group consisting of:

(m) a polynucleotide encoding RNA having a nucleotide sequence complementary to a transcript of the polynucleotide (DNA) according to (5) above;

(n) a polynucleotide encoding RNA that represses the expression of the polynucleotide (DNA) according to (5) above through an RNAi effect;

(o) a polynucleotide encoding RNA having an activity of specifically cleaving a transcript of the polynucleotide (DNA) according to (5) above; and

(p) a polynucleotide encoding RNA that represses the expression of the polynucleotide (DNA) according to (5) above through a co-suppression effect.

(q) a polynucleotide encoding RNA having a nucleotide sequence complementary to a transcript of, a polynucleotide encoding RNA that represses, through an RNAi effect, the expression of, a polynucleotide encoding RNA having an activity of specifically cleaving a transcript of, or a polynucleotide encoding RNA that represses, through a co-suppression effect, the expression of the polynucleotide (DNA) selected from:

(q1) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and having ammonia transporter activity;

(q2) a polynucleotide comprising a polynucleotide encoding a protein having 90% or higher identity with the amino acid sequence of SEQ ID NO: 4, and having ammonia transporter activity; or (q3) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, said which encodes a protein having ammonia transporter activity.

(10) A vector containing the polynucleotide according to (9) above.

(11) A yeast into which the vector according to (7), (7a), (8) or (10) above has been introduced.

(12) The yeast according to (11) above, wherein the ammonia assimilation ability is increased due to the introduction of the vector of (7), (7a) or (8) above.

(13) A yeast, wherein the expression of the polynucleotide selected from the group consisting of the polynucleotide (DNA) according to (5) above and the following polynucleotides (q1) to (q3) is repressed by:

(A) introducing the vector of (10) above;

(B) disrupting the gene according to the polynucleotide (DNA) of (5) above; (q1) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and said protein having ammonia transporter activity; (q2) a polynucleotide comprising a polynucleotide encoding a protein having 90% or higher identity with the amino acid sequence of SEQ ID NO: 4, and having ammonia transporter activity; or (q3) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, which encodes a protein having ammonia transporter activity; or

(C) introducing a mutation into a promoter or genetically altering a promoter.

(14) The yeast according to (12) above, wherein the ammonia assimilation ability is increased by increasing the expression level of the protein of (6) above.

(15) A method for producing an alcoholic beverage by using the yeast according to any one of (11) to (14) above.

(16) The method according to (15) above, wherein the brewed alcoholic beverage is a malt beverage.

(17) The method according to (15) above, wherein the brewed alcoholic beverage is wine.

(18) An alcoholic beverage produced by the method according to any one of (15) to (17) above.

(19) A method for assessing a test yeast for its ammonia assimilation ability, comprising using a primer or probe designed based on the nucleotide sequence of a gene having the nucleotide sequence of SEQ ID NO: 1 or 3 and encoding a protein having ammonia transporter activity.

(19a) A method for selecting a yeast having an enhanced ammonia assimilation ability by using the method in (18) above.

(19b) A method for producing an alcoholic beverage (for example, beer) by using the yeast selected with the method in (19a) above.

(20) A method for assessing a test yeast for its ammonia assimilation ability, comprising: culturing the test yeast; and measuring the expression level of the gene having the nucleotide sequence of SEQ ID NO: 1 or 3 and encoding a protein having ammonia transporter activity.

(20a) A method for selecting a yeast having an increased or reduced ammonia assimilation ability, which comprises assessing a test yeast by the method described in (20) above and selecting a yeast having a high expression level or low expression level of gene encoding a protein having ammonium transporter activity.

(20b) A method for producing an alcoholic beverage (for example, beer) by using the yeast selected with the method in (20a) above.

(21) A method for selecting a yeast, comprising: culturing test yeasts; quantifying the protein of (6) above or measuring the expression level of the gene having the nucleotide sequence of SEQ ID NO: 1 or 3 and encoding a protein having ammonium transporter activity; and selecting a test yeast having the production amount of the protein or the gene expression level according to the ammonium assimilation ability of interest.

(21a) A method for selecting a yeast, comprising: culturing test yeasts; measuring an ammonium assimilation ability or ammonium transporter activity; and selecting a test yeast having a target ammonium assimilation ability.

(22) The method for selecting a yeast according to (21) above, comprising: culturing a reference yeast and test yeasts; measuring for each yeast the expression level of the gene having the nucleotide sequence of SEQ ID NO: 1 or 3 and encoding a protein having ammonium transporter activity, and selecting a test yeast having the gene expression higher or lower than that in the reference yeast.

(23) The method for selecting a yeast according to (21) above, comprising: culturing a reference yeast and test yeasts; quantifying the protein according to (6) above in each yeast; and selecting a test yeast having a larger or smaller amount of the protein than that in the reference yeast.

(24) A method for producing an alcoholic beverage comprising: conducting fermentation for producing an alcoholic beverage using the yeast according to any one of (11) to (14) above or a yeast selected by the methods according to any one of (21) to (23) above, and adjusting the contents of ammonium and amino acids.

The method of producing alcoholic beverages according to this invention can control assimilation of ammonia and assimilation of amino acids, thereby controlling amino acid content of alcoholic beverages. Thus, alcoholic beverages with controlled taste can be produced.

### BRIEF DESCRIPTION OF DRAWINGS

FIG. 1 shows the cell growth with time upon beer fermentation test. The horizontal axis represents fermentation time while the vertical axis represents optical density at 660 nm (OD660).

FIG. 2 shows the extract (sugar) consumption with time upon beer fermentation test. The horizontal axis represents fermentation time while the vertical axis represents apparent extract concentration (w/w %).

FIG. 3 shows the expression profile of non-ScMEP1 gene in yeasts upon beer fermentation test. The horizontal axis represents fermentation time while the vertical axis represents the intensity of detected signal.

FIG. 4 shows the cell growth with time upon fermentation test. The horizontal axis represents fermentation time while the vertical axis represents optical density at 660 nm (OD660).

FIG. 5 shows the extract (sugar) consumption with time upon beer fermentation test. The horizontal axis represents fermentation time while the vertical axis represents apparent extract concentration (w/w %).

FIG. 6 shows ammonia concentration with time upon beer fermentation test. The horizontal axis represents fermentation time while the vertical axis represents ammonia concentration (mg/L).

FIG. 7 shows free amino nitrogen (FAN) concentration with time upon beer fermentation test. The horizontal axis represents fermentation time while the vertical axis represents free amino nitrogen (FAN) concentration (mg/100 ml).

FIG. 8 shows the expression profile of ScMEP1 gene in yeasts upon beer fermentation test. The horizontal axis represents fermentation time while the vertical axis represents the intensity of detected signal.

FIG. 9 shows the cell growth with time upon fermentation test. The horizontal axis represents fermentation time while the vertical axis represents optical density at 660 nm (OD660).

FIG. 10 shows the extract (sugar) consumption with time upon beer fermentation test. The horizontal axis represents fermentation time while the vertical axis represents apparent extract concentration (w/w %).

FIG. 11 shows ammonia concentration with time upon beer fermentation test. The horizontal axis represents fermentation time while the vertical axis represents ammonia concentration (mg/L).

FIG. 12 shows free amino nitrogen (FAN) concentration with time upon beer fermentation test. The horizontal axis represents fermentation time while the vertical axis represents free amino nitrogen (FAN) concentration (mg/100 ml).

### BEST MODES FOR CARRYING OUT THE INVENTION

The present inventors conceived that it is possible to more effectively assimilate ammonia by increasing ammonia transporter activity of the yeast. The present inventors have studied based on this conception and as a result, isolated and identified non-ScMEP1 gene encoding an ammonia transporter unique to lager brewing yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2.

Further, the present inventors isolated and identified ScMEP1 gene. The nucleotide sequence of the gene is represented by SEQ ID NO: 3. Further, an amino acid sequence of a protein encoded by the gene is represented by SEQ ID NO: 4.

### 1. Polynucleotide of the Invention

First of all, the present invention provides (a) a polynucleotide comprising a polynucleotide of the nucleotide sequence of SEQ ID NO:1 or NO:3; and (b) a polynucleotide comprising a polynucleotide encoding a protein of the amino acid sequence of SEQ ID NO:2 or NO:4. The polynucleotide can be DNA or RNA.

The target polynucleotide of the present invention is not limited to the polynucleotide encoding an ammonia transporter derived from lager brewing yeast described above and may include other polynucleotides encoding proteins having equivalent functions to said protein. Proteins with equivalent functions include, for example, (c) a protein of an amino acid sequence of SEQ ID NO:2 or NO:4 with one or more amino acids thereof being deleted, substituted, inserted and/or added and having ammonia transporter activity.

Such proteins include a protein consisting of an amino acid sequence of SEQ ID NO:2 or NO:4 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having ammonia transporter activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 790% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 990% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO:2 or NO:4, and having ammonia transporter activity. In general, the percentage identity is preferably higher.

Ammonia transporter activity may be measured, for example, by a method described in Mol Cell Biol 17:4282-93, 1997.

Furthermore, the present invention also contemplates (e) a polynucleotide comprising a polynucleotide which hybridizes to a polynucleotide consisting of a nucleotide sequence complementary to the nucleotide sequence of SEQ ID NO:1 or NO:3 under stringent conditions and which encodes a protein having ammonia transporter activity; and (f) a polynucleotide comprising a polynucleotide which hybridizes to a polynucleotide complementary to a nucleotide sequence of encoding a protein of SEQ ID NO:2 or NO:4 under stringent conditions, and which encodes a protein having ammonia transporter activity.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO:1 or NO:3 or polynucleotide encoding the amino acid sequence of SEQ ID NO:2 or NO:4, as a probe. The hybridization method may be a method described, for example, in MOLECULAR CLONING 3rd Ed., CURRENT PROTOCOLS IN MOLECULAR BIOLOGY, John Wiley & Sons 1987-1997, and so on.

The term “stringent conditions” as used herein may be any of low stringency conditions, moderate stringency conditions or high stringency conditions. “Low stringency conditions” are, for example, 5×SSC, 5× Denhardt's solution, 0.5% SDS, 50% formamide at 32° C. “Moderate stringency conditions” are, for example, 5×SSC, 5× Denhardt's solution, 0.5% SDS, 50% formamide at 42° C. “High stringency conditions” are, for example, 5×SSC, 5× Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 790% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 990% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO:2 or NO:4 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX the parameters are, for example, score=50 and word length=3. When BLAST and Gapped BLAST programs are used, default parameters for each of the programs are employed.

The polynucleotide of the present invention includes (m) a polynucleotide encoding RNA having a nucleotide sequence complementary to a transcript of the polynucleotide (DNA) according to (5) above; (n) a polynucleotide encoding RNA that represses the expression of the polynucleotide (DNA) according to (5) above through RNAi effect; (o) a polynucleotide encoding RNA having an activity of specifically cleaving a transcript of the polynucleotide (DNA) according to (5) above; and (p) a polynucleotide encoding RNA that represses expression of the polynucleotide (DNA) according to (5) above through co-suppression effect; (q) a polynucleotide encoding RNA having a nucleotide sequence complementary to a transcript of, a polynucleotide encoding RNA that represses, through an RNAi effect, the expression of, a polynucleotide encoding RNA having an activity of specifically cleaving a transcript of, or a polynucleotide encoding RNA that represses, through a co-suppression effect, the expression of the polynucleotide (DNA) selected from:

(q1) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and having ammonia transporter activity;

(q2) a polynucleotide comprising a polynucleotide encoding a protein having 90% or higher identity with the amino acid sequence of SEQ ID NO: 4, and having ammonia transporter activity; or

(q3) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, said which encodes a protein having ammonia transporter activity. These polynucleotides may be incorporated into a vector, which can be introduced into a cell for transformation to repress the expression of the polynucleotides (DNA) of (a) to (l) above. Thus, these polynucleotides may suitably be used when repression of the expression of the above DNA is preferable.

The phrase “polynucleotide encoding RNA having a nucleotide sequence complementary to the transcript of DNA” as used herein refers to so-called antisense DNA. Antisense technique is known as a method for repressing expression of a particular endogenous gene, and is described in various publications (see e.g., Hirajima and Inoue: New Biochemistry Experiment Course 2 Nucleic Acids IV Gene Replication and Expression (Japanese Biochemical Society Ed., Tokyo Kagaku Dozin Co., Ltd.) pp. 319-347, 1993). The sequence of antisense DNA is preferably complementary to all or part of the endogenous gene, but may not be completely complementary as long as it can effectively repress the expression of the gene. The transcribed RNA has preferably 90% or higher, and more preferably 95% or higher complementarity to the transcript of the target gene. The length of the antisense DNA is at least 15 bases or more, preferably 100 bases or more, and more preferably 500 bases or more.

The phrase “polynucleotide encoding RNA that represses DNA expression through RNAi effect” as used herein refers to a polynucleotide for repressing expression of an endogenous gene through RNA interference (RNAi). The term “RNAi” refers to a phenomenon where when double-stranded RNA having a sequence identical or similar to the target gene sequence is introduced into a cell, the expressions of both the introduced foreign gene and the target endogenous gene are repressed. RNA as used herein includes, for example, double-stranded RNA that causes RNA interference of 21 to 25 base length, for example, dsRNA (double strand RNA), siRNA (small interfering RNA) or shRNA (short hairpin RNA). Such RNA may be locally delivered to a desired site with a delivery system such as liposome, or a vector that generates the double-stranded RNA described above may be used for local expression thereof. Methods for producing or using such double-stranded RNA (dsRNA, siRNA or shRNA) are known from many publications (see, e.g., Japanese National Phase PCT Laid-open Patent Publication No. 2002-516062; US 2002/086356A; Nature Genetics, 24(2), 180-183, 2000 February; Genesis, 26(4), 240-244, 2000 April; Nature, 407:6802, 319-20, 2002 Sep. 21; Genes & Dev., Vol. 16, (8), 948-958, 2002 Apr. 15; Proc. Natl. Acad. Sci. USA., 99(8), 5515-5520, 2002 Apr. 16; Science, 296(5567), 550-553, 2002 Apr. 19; Proc Natl Acad. Sci. USA, 99:9, 6047-6052, 2002 Apr. 30; Nature Biotechnology, Vol. 20 (5), 497-500, 2002 May; Nature Biotechnology, Vol. 20(5), 500-505, 2002 May; Nucleic Acids Res., 30:10, e46, 2002 May 15).

The phrase “polynucleotide encoding RNA having an activity of specifically cleaving transcript of DNA” as used herein generally refers to a ribozyme. Ribozyme is an RNA molecule with a catalytic activity that cleaves a transcript of a target DNA and inhibits the function of that gene. Design of ribozymes can be found in various known publications (see, e.g., FEBS Lett. 228: 228, 1988; FEBS Lett. 239: 285, 1988; Nucl. Acids. Res. 17: 7059, 1989; Nature 323: 349, 1986; Nucl. Acids. Res. 19:6751, 1991; Protein Eng 3: 733, 1990; Nucl. Acids Res. 19:3875, 1991; Nucl. Acids Res. 19: 5125, 1991; Biochem Biophys Res Commun 186: 1271, 1992). In addition, the phrase “polynucleotide encoding RNA that represses DNA expression through co-supression effect” refers to a nucleotide that inhibits functions of target DNA by “co-supression”.

The term “co-supression” as used herein, refers to a phenomenon where when a gene having a sequence identical or similar to a target endogenous gene is transformed into a cell, the expressions of both the introduced foreign gene and the target endogenous gene are repressed. Design of polynucleotides having a co-supression effect can also be found in various publications (see, e.g., Smyth D R: Curr. Biol. 7: R793, 1997, Martienssen R: Curr. Biol. 6: 810, 1996).

### 2. Protein of the Present Invention

The present invention also provides proteins encoded by any of the polynucleotides (a) to (e) above. A preferred protein of the present invention comprises an amino acid sequence of SEQ ID NO:2 or NO:4 with one or several amino acids thereof being deleted, substituted, inserted and/or added, and has ammonia transporter activity.

Such protein includes those having an amino acid sequence of SEQ ID NO:2 or NO:4 with amino acid residues thereof of the number mentioned above being deleted, substituted, inserted and/or added and having ammonia transporter activity. In addition, such protein includes those having homology as described above with the amino acid sequence of SEQ ID NO:2 or NO:4 and having ammonia transporter activity.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (q) above. Generally, the vector of the present invention comprises an expression cassette including as components (x) a promoter that can transcribe in a yeast cell; (y) a polynucleotide described in any of (a) to (q) above that is linked to the promoter in sense or antisense direction; and (z) a signal that functions in the yeast with respect to transcription termination and polyadenylation of RNA molecule. According to the present invention, in order to highly express the protein of the invention described above upon brewing alcoholic beverages (e.g., beer) described below, these polynucleotides are introduced in the sense direction to the promoter to promote expression of the polynucleotide (DNA) described in any of (a) to (l) above. Further, in order to repress the above protein of the invention upon brewing alcoholic beverages (e.g., beer) described below, these polynucleotides are introduced in the antisense direction to the promoter to repress the expression of the polynucleotide (DNA) described in any of (a) to (l) above. In order to repress the above protein of the invention, the polynucleotide may be introduced into vectors such that the polynucleotide of any of the (m) to (q) is to be expressed. According to the present invention, the target gene (DNA) may be disrupted to repress the expression of the DNA described above or the expression of the protein described above. A gene may be disrupted by adding or deleting one or more bases to or from a region involved in expression of the gene product in the target gene, for example, a coding region or a promoter region, or by deleting these regions entirely. Such disruption of gene may be found in known publications (see, e.g., Proc. Natl. Acad. Sci. USA, 76, 4951 (1979), Methods in Enzymology, 101, 202 (1983), Japanese Patent Application Laid-Open No. 6-253826).

Further, in the present invention, the expression level of a target gene can be controlled by introducing a mutation to a promoter or genetically altering a promoter by homologous recombination. Such mutation introducing method is described in Nucleic Acids Res. 29, 4238-4250 (2001), and such alteration of a promoter is described in, for example, Appl Environ Microbiol., 72, 5266-5273 (2006).

A vector introduced in the yeast may be any of a multicopy type (YEp type), a single copy type (YCp type), or a chromosome integration type (YIp type). For example, YEp24 (J. R Broach et al., EXPERIMENTAL MANIPULATION OF GENE EXPRESSION, Academic Press, New York, 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they are not influenced by constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yeasts such as genus Saccharomyces may be used. According to the present invention, a lager brewing yeast, for example, Saccharomyces pastorianus W34/70, etc., Saccharomyces carlsbergensis NCYC453 or NCYC456, etc., or Saccharomyces cerevisiae NBRC1951, NBRC1952, NBRC1953 or NBRC1954, etc., may be used. In addition, whisky yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering: Vol. 1, Plenum Press, New York, 117 (1979)), etc.) such that OD600 nm will be 1 to 6. This culture yeast is collected by centrifugation, washed and pre-treated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed., and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to produce alcoholic beverages with controlled ammonia content and controlled amino acid content. In addition, yeasts to be selected by the yeast assessment method of the present invention described below can also be used. The target alcoholic beverages include, for example, but not limited to beer, beer-taste beverages such as sparkling liquor (happoushu), wine, whisky, sake and the like. Further, according to the present invention, desired alcoholic beverages with increased ammonia level can be produced using brewery yeast in which the expression of the target gene was suppressed, if needed. That is to say, desired kind of alcoholic beverages with controlled (elevated or reduced) level of ammonia can be produced by controlling (elevating or reducing) production amount of ammonia using yeasts into which the vector of the present invention was introduced described above, yeasts in which expression of the polynucleotide (DNA) of the present invention described above was suppressed or yeasts selected by the yeast assessment method of the invention described below for fermentation to produce alcoholic beverages.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages. Thus, according to the present invention, alcoholic beverages can be produced using the existing facility without increasing the cost.

### 5. Yeast Assessment Method of the Invention

The present invention relates to a method for assessing a test yeast for its ammonia assimilation ability by using a primer or a probe designed based on a nucleotide sequence of an ammonia transporter gene having the nucleotide sequence of SEQ ID NO:1 or NO:3. General techniques for such assessment method is known and is described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the ammonia transporter gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the ammonia assimilation ability of the yeast as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the gene encoding a protein having ammonia transporter activity having the nucleotide sequence of SEQ ID NO:1 or NO:3 to assess the test yeast for its ammonia assimilation ability. In measuring an expression level of the gene encoding a protein having ammonia transporter activity, the test yeast is cultured and then mRNA or a protein resulting from the ammonia transporter gene is quantified. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS IN MOLECULAR BIOLOGY, John Wiley & Sons 1994-2003). Further, expression level of the above-described gene of the test yeast may be projected by measuring ammonia concentration of fermentation broth obtained after fermentation of the test yeast.

Furthermore, test yeasts are cultured and expression levels of the gene encoding a protein having ammonia transporter activity having the nucleotide sequence of SEQ ID NO:1 or NO:3 are measured to select a test yeast with the gene expression level according to the target ammonia transporter activity, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the gene encoding a protein having ammonia transporter activity having the nucleotide sequence of SEQ ID NO:1 or NO:3 is measured in each yeast. By selecting a test yeast with the gene expressed higher or lower than that in the reference yeast, a yeast suitable for brewing alcoholic beverages can be selected.

Alternatively, test yeasts are cultured and a yeast with a higher or lower ammonia transporter activity is selected, thereby selecting a yeast suitable for brewing desired alcoholic beverages.

In these cases, the test yeasts or the reference yeast may be, for example, a yeast introduced with the vector of the invention, a yeast in which an expression of a polynucleotide (DNA) of the invention has been controlled, an artificially mutated yeast or a naturally mutated yeast. The ammonia transporter activity can be measured by, for example, a method described in Mol Cell Biol 17:4282-93, 1997. The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., BIOCHEMISTRY EXPERIMENTS vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954, etc., may be used. Further, wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Ammonia Transporter (NonScMEP1)

A specific ammonia transporter gene (nonScMEP1) (SEQ ID NO:1) from a lager brewing yeast has been found, as a result of a search utilizing the comparison database described in Japanese Patent Application Laid-Open No. 2004-283169. Based on the nucleotide sequence information acquired, primers nonScMEP1_F (SEQ ID NO: 5) and nonScMEP1_R (SEQ ID NO: 6) were designed to amplify the full-length of the gene. PCR was carried out using chromosomal DNA of a genome sequencing strain, Saccharomyces pastorianus Weihenstephan 34/70 (may be abbreviated as “W34/79 strain”), as a template to obtain DNA fragments including the full-length gene for nonScMEP1.

The nonScMEP1 gene fragments thus obtained were inserted into pCR2.1-TOPO vector (Invitrogen) by TA cloning. The nucleotide sequences for the nonScMEP1 gene were analyzed by Sanger's method (F. Sanger, Science, 214: 1215, 1981) to confirm the nucleotide sequence.

### Example 2

### Analysis of Expression of nonScMEP1 Gene During Beer Fermentation

A beer fermentation test was conducted using a lager brewing yeast, Saccharomyces pastorianus W34/70, and mRNA extracted from the yeast cells during fermentation was analyzed by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

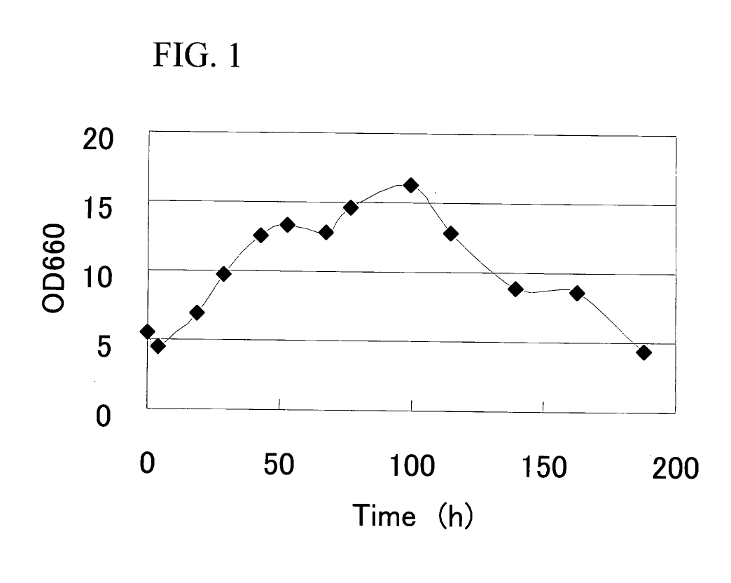

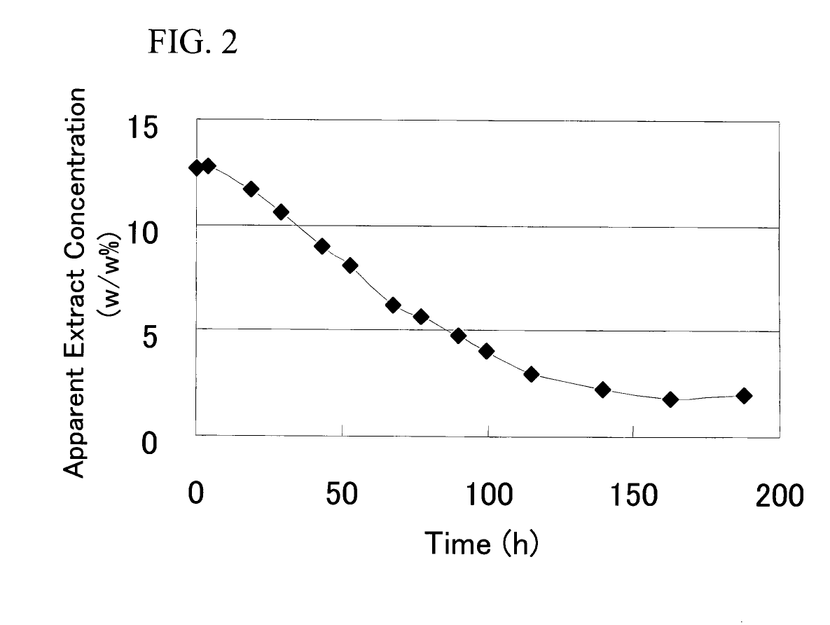

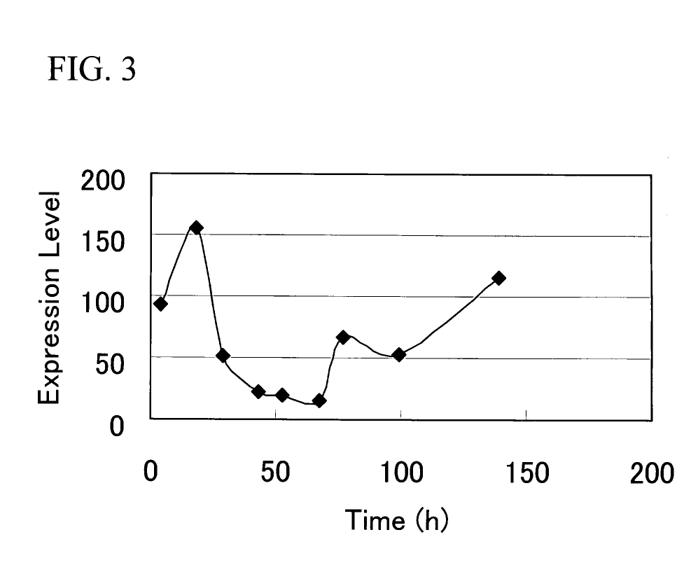

The fermentation liquor was sampled over time, and the time-course changes in amount of yeast cell growth (FIG. 1) and apparent extract concentration (FIG. 2) were observed. Simultaneously, sampling of yeast cells was performed, and the prepared mRNA was biotin-labeled and subjected to hybridization with the beer yeast DNA microarray disclosed in Japanese Patent Application Laid-Open No. 2004-283169. Signal detection was performed using the GeneChip Operating system (GCOS; GeneChip Operating Software 1.0, manufactured by Affymetrix Co). Expression pattern of the nonScMEP1 gene is shown in FIG. 3. This result confirmed the expression of the nonScMEP1 gene in the conventional beer fermentation.

### Example 3

### Construction of NonScMEP1-Highly Expressed Strain

The nonScMEP1/pCR2.1-TOPO described in Example 1 was digested with the restriction enzymes SacI and NotI to prepare a DNA fragment containing the entire length of the protein-encoding region. This fragment was ligated to pYCGPYNot treated with the restriction enzymes SacI and NotI, thereby constructing the nonScMEP1 high expression vector nonScMEP1/pYCGPYNot. pYCGPYNot is a YCp-type yeast expression vector. A gene inserted is highly expressed by the pyruvate kinase gene PYK1 promoter. The geneticin-resistant gene G418r is included as the selectable marker in the yeast, and the ampicillin-resistant gene Amp^{r} as the selectable marker in Escherichia coli.

Using the high expression vector prepared by the above method, the strain Saccharomyces pasteurianus Weihenstephaner 34/70 was transformed by the method described in Japanese Patent Application Laid-open No. 7-303475. The transformants were selected on a YPD plate culture (1% yeast extract, 2% polypeptone, 2% glucose and 2% agar) containing 300 mg/L of geneticin.

### Example 4

### Measurement of Assimilation of Ammonia and Amino Acids in Beer Fermentation

A fermentation test was carried out under the following conditions using the parent strain and the nonScMEP1-highly expressed strain obtained in Example 3.

| | |
|---|---|
| Wort extract concentration | 12% |
| Wort content | 1 L |
| Wort dissolved oxygen concentration | approx. 8 ppm |
| Fermentation temperature | 15° C. (fixed) |
| Yeast pitching rate | 5 g wet yeast cells/L of wort |

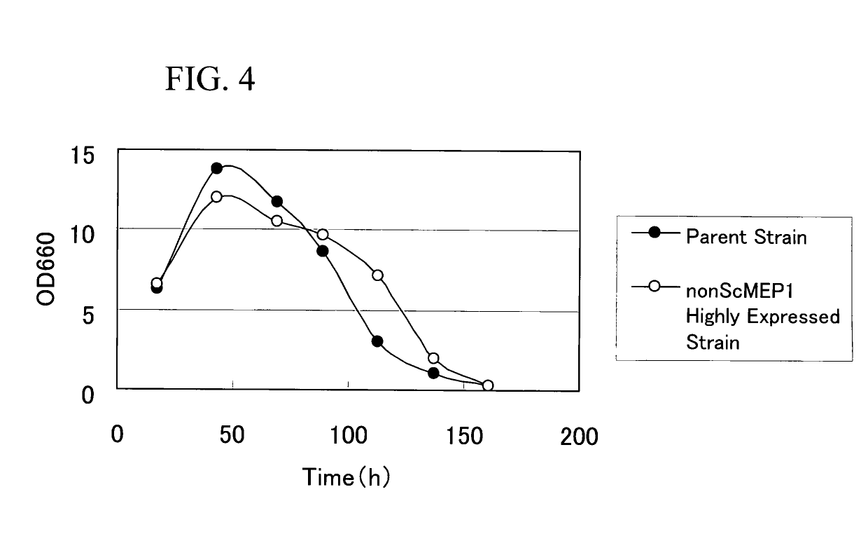

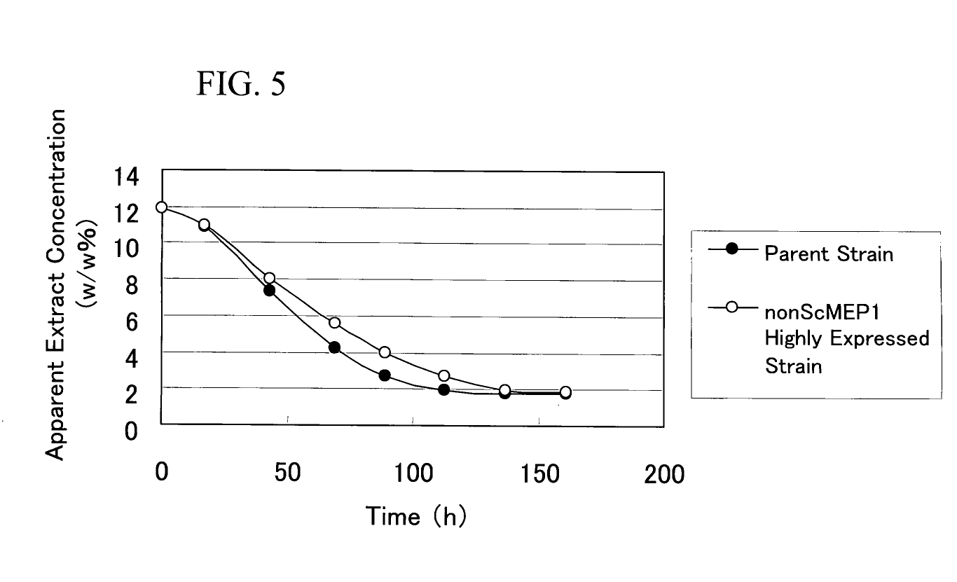

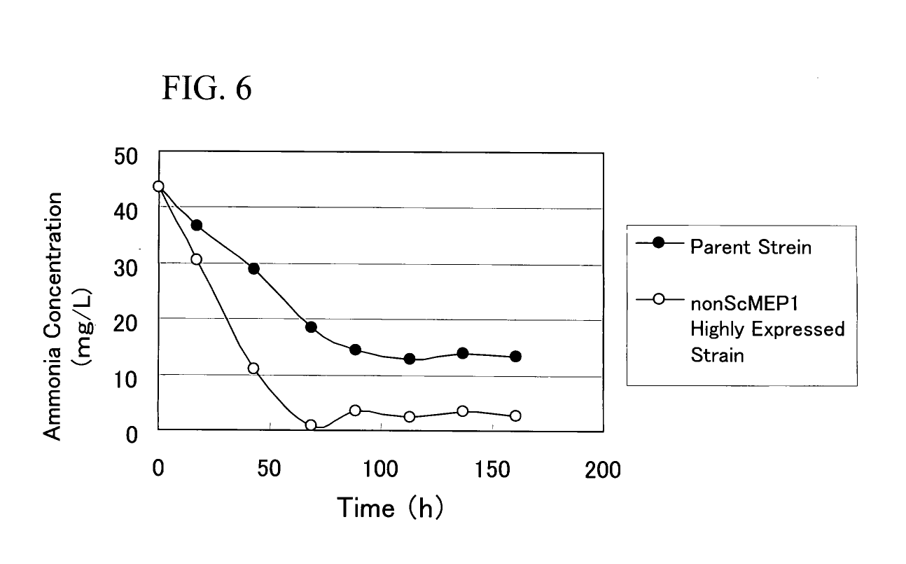

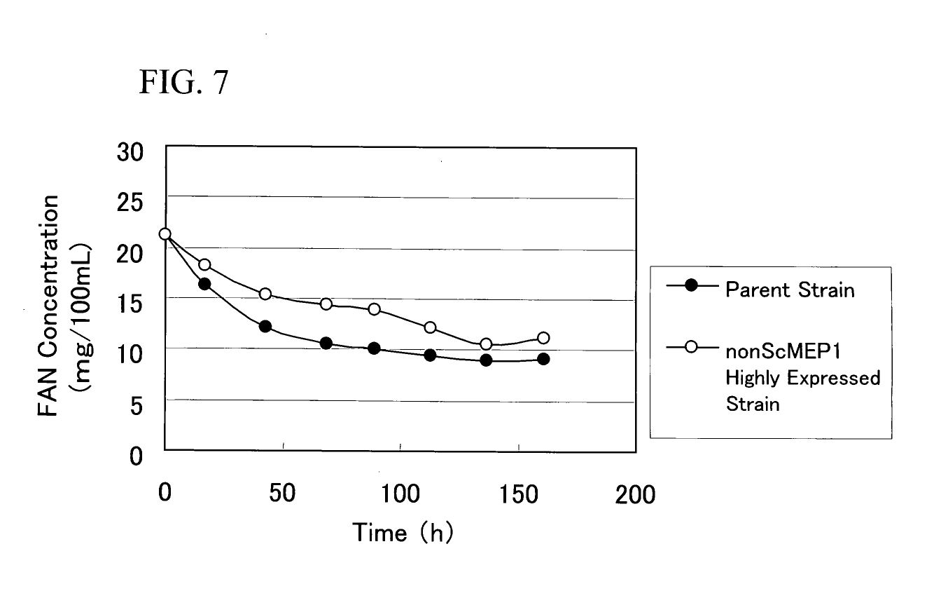

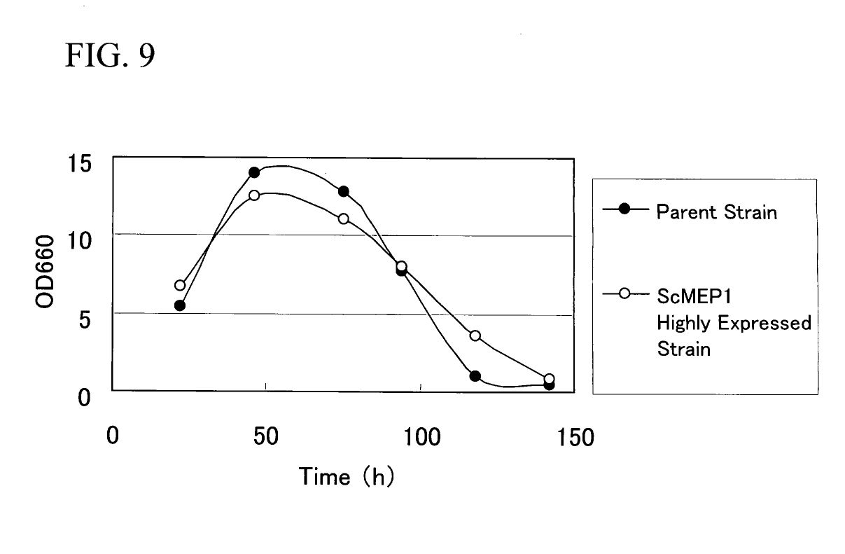

The fermentation broth was sampled over time, and the time-course changes in yeast cell growth rate (OD660) (FIG. 4), the amount of extract consumption (FIG. 5), the ammonia concentration (FIG. 6), and the free amino nitrogen concentration (FIG. 9) were observed. While the ammonia assimilation was enhanced in the nonScMEP1-highly expressed strain as compared to the parent strain, as shown in FIG. 6, the assimilation of free amino nitrogen (FAN) was reduced as shown in FIG. 7. As a result, as shown in Table 1, the amount of total amino acids was increased but the amount of ammonia was decreased in the beer at the completion of fermentation of the nonScMEP1-highly expressed strain

TABLE 1

| | | nonScMEP1-Highly |
|---|---|---|
| Amino Acid Concentration (mM) | Parent Strain | Expressed Strain |
| Aspartic acid | 0.0056 | 0.0136 |
| Threonine | 0.0552 | 0.0488 |
| Serine | Not Detected | Not Detected |
| Glutamic Acid | 0.0998 | 0.2340 |
| Glycine | 0.2385 | 0.3284 |
| Alanine | 0.6723 | 0.9905 |
| Cystein | 0.0133 | 0.0154 |
| Valine | 0.3689 | 0.5476 |
| Methionine | 0.0056 | Not Detected |
| Isoleucine | 0.0438 | 0.1186 |
| Leucine | 0.0753 | 0.2003 |
| Tyrosine | 0.2918 | 0.3503 |
| Phenylalanine | 0.3599 | 0.4798 |
| Ornithine | 0.0428 | 0.0462 |
| Lysine | 0.1036 | 0.1217 |
| Histidine | 0.1225 | 0.1951 |
| Tryptophan | 0.1387 | 0.1149 |
| Arginine | 0.0088 | 0.2014 |
| Proline | 3.9525 | 4.1067 |
| Total Amino Acids(mM) | 6.5985 | 8.1128 |
| Ammonia(mg/L) | 13.464 | 2.693 |

### Example 5

### Cloning of Ammonia Transporter (ScMEP1)

A specific ammonia transporter gene (ScMEP1) (SEQ ID NO: 3) from a lager brewing yeast has been found, as a result of a search utilizing the comparison database described in Japanese Patent Application Laid-Open No. 2004-283169. Based on the nucleotide sequence information acquired, primers ScMEP1_F (SEQ ID NO: 7) and ScMEP1_R (SEQ ID NO: 8) were designed to amplify the full-length of the gene. PCR was carried out using chromosomal DNA of a genome sequencing strain, Saccharomyces pastorianus Weihenstephan 34/70, as a template to obtain DNA fragments including the full-length gene for ScMEP1.

The ScMEP1 gene fragments thus obtained were inserted into pCR2.1-TOPO vector (Invitrogen) by TA cloning. The nucleotide sequences for the ScMEP1 gene were analyzed by Sanger's method (F. Sanger, Science, 214: 1215, 1981) to confirm the nucleotide sequence.

### Example 6

### Analysis of Expression of ScMEP1 Gene During Beer Fermentation

A beer fermentation test was conducted using a lager brewing yeast, Saccharomyces pastorianus W34/70, and mRNA extracted from the yeast cells during fermentation was analyzed by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

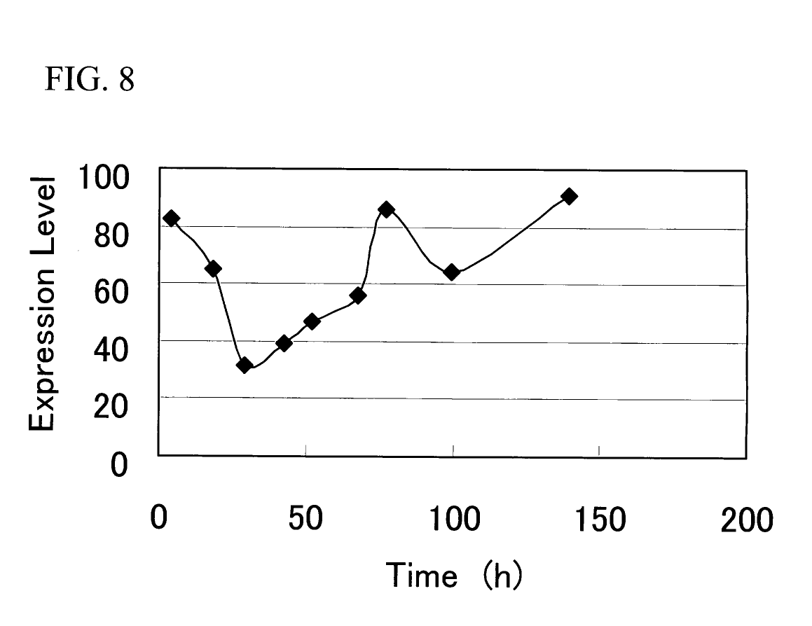

The fermentation liquor was sampled over time, and the time-course changes in amount of yeast cell growth (FIG. 1) and apparent extract concentration (FIG. 2) were observed. Simultaneously, sampling of yeast cells was performed, and the prepared mRNA was biotin-labeled and subjected to hybridization with the yeast DNA microarray disclosed in Japanese Patent Application Laid-Open No. 2004-283169. Signal detection was performed using the GeneChip Operating system (GCOS; GeneChip Operating Software 1.0, manufactured by Affymetrix Co). Expression pattern of the ScMEP1 gene is shown in FIG. 8. This result confirmed the expression of the ScMEP1 gene in the conventional beer fermentation.

### Example 7

### Construction of ScMEP1-Highly Expressed Strain

The ScMEP1/pCR2.1-TOPO described in Example 1 was digested with the restriction enzymes SacI and NotI to prepare a DNA fragment containing the entire length of the protein-encoding region. This fragment was ligated to pYCGPYNot treated with the restriction enzymes SacI and NotL thereby constructing the ScMEP1 high expression vector ScMEP1/pYCGPYNot. pYCGPYNot is a YCp-type yeast expression vector. A gene inserted is highly expressed by the pyruvate kinase gene PYK1 promoter. The geneticin-resistant gene G418^{r} is included as the selectable marker in the yeast, and the ampicillin-resistant gene Amp^{r} as the selectable marker in Escherichia coli.

Using the high expression vector prepared by the above method, the strain Saccharomyces pasteurianus Weihenstephaner 34/70 was transformed by the method described in Japanese Patent Application Laid-open No. 7-303475. The transformants were selected on a YPD plate culture (1% yeast extract, 2% polypeptone, 2% glucose and 2% agar) containing 300 mg/L of geneticin.

### Example 8

### Measurement of Assimilation of Ammonia and Amino Acids in Beer Fermentation

A fermentation test was carried out under the following conditions using the parent strain and the ScMEP1-highly expressed strain obtained in Example 7.

| | |
|---|---|
| Wort extract concentration | 12% |
| Wort content | 1 L |
| Wort dissolved oxygen concentration | approx. 8 ppm |
| Fermentation temperature | 15° C. (fixed) |
| Yeast pitching rate | 5 g wet yeast cells/L of wort |

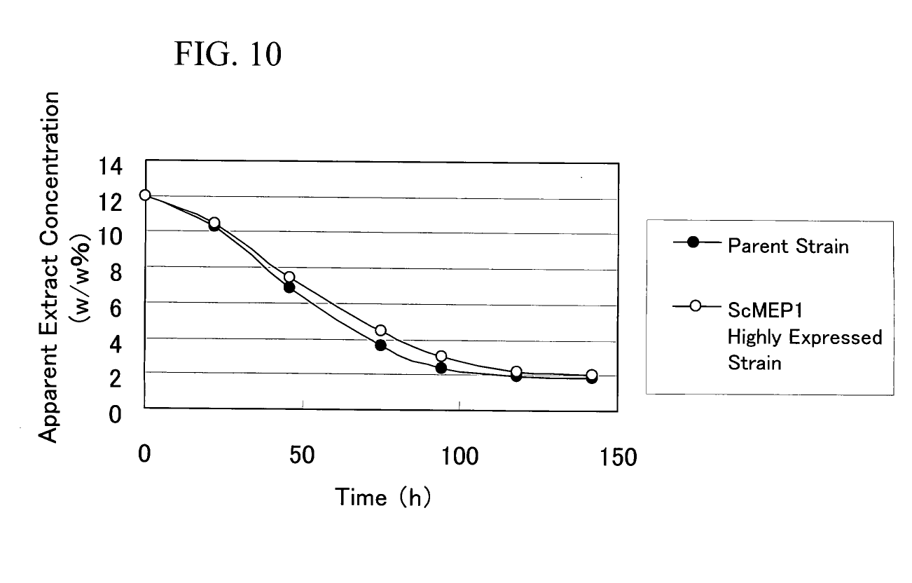

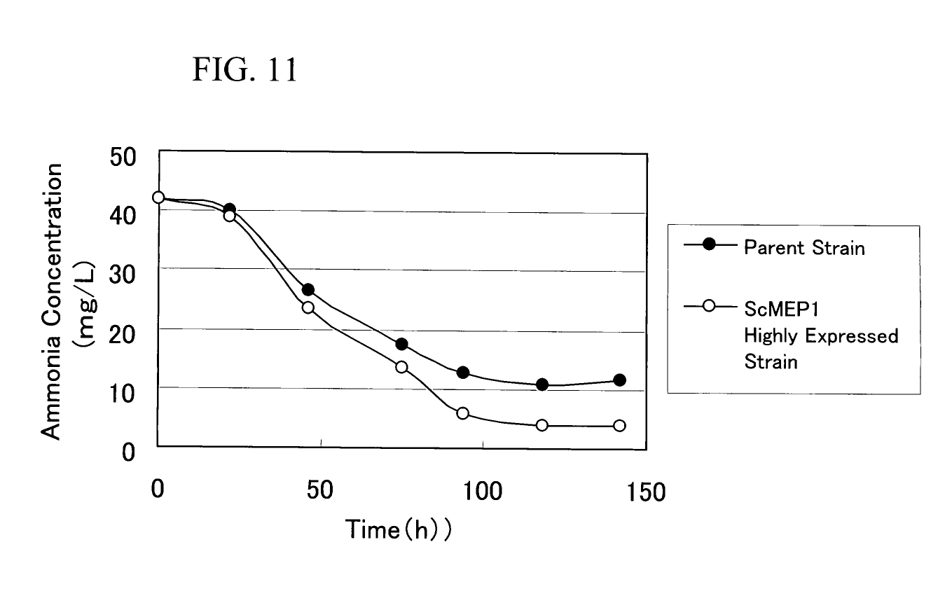

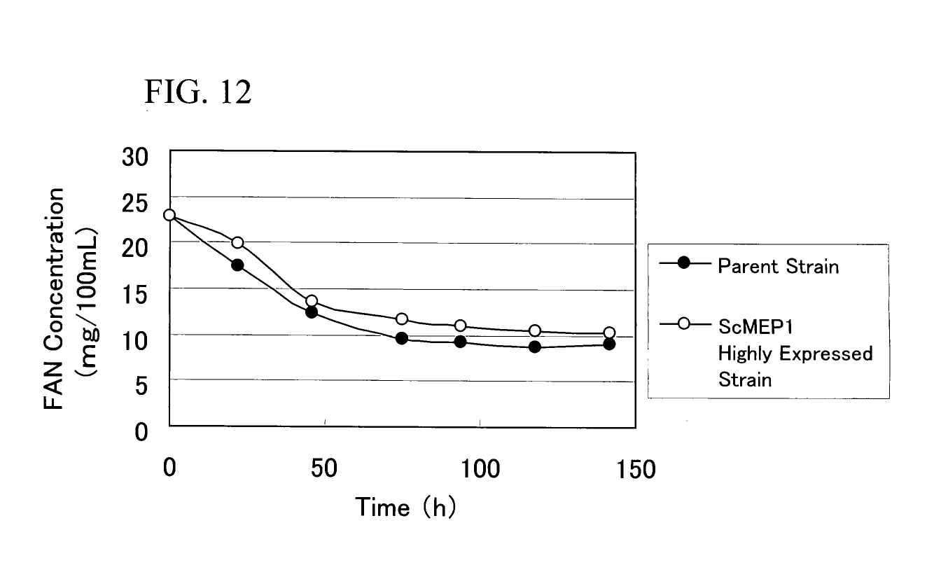

The fermentation broth was sampled over time, and the time-course changes in yeast cell growth rate (OD660) (FIG. 9), the amount of extract consumption (FIG. 10), the ammonia concentration (FIG. 11), and the free amino nitrogen concentration (FIG. 12) were observed. While the ammonia assimilation was enhanced in the ScMEP1-highly expressed strain as compared to the parent strain, as shown in FIG. 11, the assimilation of free amino nitrogen (FAN) was reduced as shown in FIG. 12. As a result, as shown in Table 2, the amount of total amino acids was increased but the amount of ammonia was decreased in the beer at the completion of fermentation of the ScMEP1-highly expressed strain

TABLE 2

| | | ScMEP1-Highly |
|---|---|---|
| Amino Acid Concentration (mM) | Parent Strain | Expressed Strain |
| Aspartic acid | Not Detected | Not Detected |
| Threonine | 0.0488 | Not Detected |
| Serine | Not Detected | 0.0165 |
| Glutamic Acid | 0.0907 | 0.1958 |
| Glycine | 0.2338 | 0.3081 |
| Alanine | 0.6629 | 0.9336 |
| Cystein | 0.0178 | 0.0206 |
| Valine | 0.3654 | 0.4802 |
| Methionine | Not Detected | Not Detected |
| Isoleucine | 0.0585 | 0.1074 |
| Leucine | 0.0800 | 0.1677 |
| Tyrosine | 0.4759 | 0.4871 |
| Phenylalanine | 0.3432 | 0.4060 |
| Ornithine | 0.0370 | 0.0430 |
| Lysine | Not Detected | Not Detected |
| Histidine | 0.1154 | 0.1882 |
| Tryptophan | 0.1354 | 0.1641 |
| Arginine | Not Detected | 0.0541 |
| Proline | 4.0057 | 4.0504 |
| Total Amino Acids(mM) | 6.6701 | 7.6225 |
| Ammonia(mg/L) | 11.590 | 3.750 |

### Example 9

### Disruption of NonScMEP1 or ScMEP1 Gene

According to the method described in the publication (Goldstein et al., yeast. 15 1541 (1999)), a fragment for gene disruption is prepared by PCR using a plasmid containing a drug-resistance marker (pFA6a (G418^{r}), pAG25 (nat1) or pAG32 (hph)) as a template.

The fragment for gene disruption thus prepared is used to transform the W34/70 strain or the spore cloning strain W34/70-2. The transformation is performed in accordance with the method described in Japanese Patent Application Laid-Open No. 7-303475. The concentrations of the drugs for selection are 300 mg/L for geneticin and 50 mg/L for nourseothricin.

### Example 10

### Measurement of Assimilation of Ammonia and Amino Acids in Beer Fermentation

Using the parent strain and the nonScMEP1-disrupted strain or the ScMEP1-disrupted strain obtained in Example 9, fermentation test is carried out under the following conditions.

| | |
|---|---|
| Wort extract concentration | 12% |
| Wort content | 1 L |
| Wort dissolved oxygen concentration | about 7 ppm |
| Fermentation temperature | 12° C. constantly |
| Yeast pitching rate | 5 g of wet yeast cells/L of wort |

Likewise in Example 8, the fermentation broth is sampled over time to observe the time course changes in cell growth (OD660), extract consumption, ammonia concentration and free amino nitrogen (FAN) concentration.

### INDUSTRIAL APPLICABILITY

The inventive method of producing alcoholic beverages may allow for production of alcoholic beverages with high amino acid content, because the ability of yeast to assimilate ammonia is enhanced and the assimilation of amino acids is reduced by the method of the present invention.

1. A polynucleotide selected from the group consisting of:
(a) a polynucleotide comprising a polynucleotide consisting of the nucleotide sequence of SEQ ID NO:1;
(b) a polynucleotide comprising a polynucleotide encoding a protein consisting of the amino acid sequence of SEQ ID NO:2;
(c) a polynucleotide comprising a polynucleotide encoding a protein consisting of the amino acid sequence of SEQ ID NO:2 in which one or more amino acids thereof are deleted, substituted, inserted and/or added, and having ammonia transporter activity;
(d) a polynucleotide comprising a polynucleotide encoding a protein having an amino acid sequence having 60% or higher identity with the amino acid sequence of SEQ ID NO:2, and said protein having ammonia transporter activity;
(e) a polynucleotide comprising a polynucleotide which hybridizes to a polynucleotide consisting of a nucleotide sequence complementary to the nucleotide sequence of SEQ ID NO:1 under stringent conditions, and which encodes a protein having ammonia transporter activity; and
(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO:2 under stringent conditions, and which encodes a protein having ammonia transporter activity.

2. The polynucleotide according to claim 1 selected from the group consisting of:
(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has ammonia transporter activity;
(h) a polynucleotide comprising a polynucleotide encoding a protein having 90% or higher identity with the amino acid sequence of SEQ ID NO: 2, and having ammonia transporter activity; and
(i) a polynucleotide comprising a polynucleotide which hybridizes to a polynucleotide consisting of a nucleotide sequence of SEQ ID NO: 1 or which hybridizes to a polynucleotide consisting of a nucleotide sequence complementary to the nucleotide sequence of SEQ ID NO: 1, under high stringent conditions, which encodes a protein having ammonia transporter activity.

3. The polynucleotide according to claim 1 comprising a polynucleotide consisting of the nucleotide sequence of SEQ ID NO: 1.

4. The polynucleotide according to claim 1 comprising a polynucleotide encoding a protein consisting of the amino acid sequence of SEQ ID NO: 2.

5. The polynucleotide according to claim 1, wherein the polynucleotide is DNA.

6. A protein encoded by the polynucleotide according to claim 1.

7. A vector containing the polynucleotide according to claim 1.

8. A vector containing any one of the polynucleotides selected from the group consisting of:
(j) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and having ammonia transporter activity;
(k) a polynucleotide comprising a polynucleotide encoding a protein having 90% or higher identity with the amino acid sequence of SEQ ID NO: 4, and having ammonia transporter activity; and
(l) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, which encodes a protein having ammonia transporter activity.

9. A polynucleotide selected from the group consisting of:
(m) a polynucleotide encoding RNA having a nucleotide sequence complementary to a transcript of the polynucleotide (DNA) according to claim 5;
(n) a polynucleotide encoding RNA that represses the expression of the polynucleotide (DNA) according to claim 5 through an RNAi effect;
(o) a polynucleotide encoding RNA having an activity of specifically cleaving a transcript of the polynucleotide (DNA) according to claim 5; and
(p) a polynucleotide encoding RNA that represses the expression of the polynucleotide (DNA) according to claim 5 through a co-suppression effect.
(q) a polynucleotide encoding RNA having a nucleotide sequence complementary to a transcript of, a polynucleotide encoding RNA that represses, through an RNAi effect, the expression of, a polynucleotide encoding RNA having an activity of specifically cleaving a transcript of, or a polynucleotide encoding RNA that represses, through a co-suppression effect, the expression of the polynucleotide (DNA) selected from:
(q1) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and having ammonia transporter activity;
(q2) a polynucleotide comprising a polynucleotide encoding a protein having 90% or higher identity with the amino acid sequence of SEQ ID NO: 4, and having ammonia transporter activity; or
(q3) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, said which encodes a protein having ammonia transporter activity.

10. A vector containing the polynucleotide according to claim 9.

11. A yeast into which the vector according to claim 7 has been introduced.

12. The yeast according to claim 11, wherein the ammonia assimilation ability is increased owing to the introduction of the vector containing the polynucleotide.

13. A yeast, wherein the expression of the polynucleotide selected from the group consisting of the polynucleotide (DNA) according to claim 5 and the following polynucleotides (q1) to (q3) is repressed by:
(A) introducing the vector containing the polynucleotide;
(B) disrupting the gene according to the polynucleotide (DNA) of claim 5; (q1) a polynucleotide comprising a polynucleotide encoding a protein consisting of the amino acid sequence of SEQ ID NO: 4, or encoding the amino acid sequence of SEQ ID NO: 4 in which 1 to 10 amino acids are deleted, substituted, inserted, and/or added, and said protein having ammonia transporter activity; (q2) a polynucleotide comprising a polynucleotide encoding a protein having 90% or higher identity with the amino acid sequence of SEQ ID NO: 4, and having ammonia transporter activity; or (q3) a polynucleotide comprising a polynucleotide which hybridizes to the polynucleotide consisting of a nucleotide sequence of SEQ ID NO: 3 or which hybridizes to a polynucleotide consisting of a nucleotide sequence complementary to the nucleotide sequence of SEQ ID NO: 3, under high stringent conditions, which encodes a protein having ammonia transporter activity; or
(C) introducing a mutation into a promoter or genetically altering a promoter.

14. The yeast according to claim 12, wherein the ammonia assimilation ability is increased by increasing the expression level of the protein encoded by the polynucleotide.

15. A method for producing an alcoholic beverage by using the yeast according to claim 11.

16. The method according to claim 15, wherein the brewed alcoholic beverage is a malt beverage.

17. The method according to claim 15, wherein the brewed alcoholic beverage is wine.

18. An alcoholic beverage produced by the method according claim 15.

19. A method for assessing a test yeast for its ammonia assimilation ability, comprising using a primer or probe designed based on the nucleotide sequence of a gene having the nucleotide sequence of SEQ ID NO: 1 or 3 and encoding a protein having ammonia transporter activity.

20. A method for assessing a test yeast for its ammonia assimilation ability, comprising: culturing the test yeast; and measuring the expression level of the gene having the nucleotide sequence of SEQ ID NO: 1 or 3 and encoding a protein having ammonia transporter activity.

21. A method for selecting a yeast, comprising: culturing test yeasts; quantifying the protein of claim 6 or measuring the expression level of the gene having the nucleotide sequence of SEQ ID NO: 1 or 3 and encoding a protein having ammonia transporter activity; and selecting a test yeast having the production amount of the protein or the gene expression level according to the ammonia assimilation ability of interest.

22. The method for selecting a yeast according to claim 21, comprising: culturing a reference yeast and test yeasts; measuring for each yeast the expression level of the gene having the nucleotide sequence of SEQ ID NO: 1 or 3 and encoding a protein having ammonia transporter activity; and selecting a test yeast having the gene expression higher or lower than that in the reference yeast.

23. The method for selecting a yeast according to claim 21, comprising: culturing a reference yeast and test yeasts; quantifying the protein encoded by the polynucleotide in each yeast; and selecting a test yeast having a larger or smaller amount of the protein than that in the reference yeast.

24. A method for producing an alcoholic beverage comprising: conducting fermentation for producing an alcoholic beverage using the yeast according to claim 11 or a yeast selected by the methods according to claim 21, and adjusting the contents of ammonia and amino acids.


SEQUENCE LISTING (8 sequences)

SEQ ID NO: 1 - DNA, 1476 bp, Saccharomyces sp.
atggaaagtcgatctacagggcctcttacgacggaaatttacgatggccccactgtggcctttatgattttgggtgccgccttggtttttttcatggtacctgggctgggattcttgtactcaggactggcgagaagaaaatccgcattggcgttgatttgggtagtgctgatggcgacgctggtcggtatactgcaatggtacttttggggttactctttagccttttccaagtccgccaccaacaacaaattcattggaaacttggactcgtttgggtttaggaacgtatacgggaagaaatccgacggcgatgtctatcctgaacttgcgtatgcgaccttccagatgatgttttcctgcgtcaacttaagcattatcgcaggcgctacagctgaaagaggtagattgctaccgcacatggttttcctcttcattctagccactattgtgtactgtccggtgacgtactggatttggtctccgggcggttgggcttaccaatggggagtgctggactgggcaggcggtgggaacattgagattctgagtgccgtttccggattcgtctactcttggtttttaggcaaaagaaatgaaaaactgctgataaacttcagacctcataatgtgtcgttggtcactctaggcacgtccatactgtggtttggttggttactcttcaattcagcctcgtcactatcaccaaatttgaggtcagtttatgcctttatgaacacgtgcctcagcgccattactggcgggatgacatggtgcctcttggattatagatcagaaaggaaatggtcgactgtcggtttgtgttctggtatcatctcaggattggtagcagccacaccaagttcaggatgtataactctctacggctcgctaatccaaggcattgtggcggggatcgtctgtaattttgccactaaattaaaatactatgctaaagtggatgacgccatggatattcttgctgagcacggagtcgcaggcataataggactaatattcaatgctcttttcgcggcagattgggtgatcggtatggacggagtcagcgaacacgagggtggctggataagtcataattacaagcaaatgtacaagcagattgcttatattgccgcatcaataggatacactgccgttgtcacggcaatcatttgctttgtgctcgggtacattcctggaatgacgctgagaatatctgatgaagcagaagaacgtggtatggatgaagaccaaattggtgaattcgcatacgactatgtggaggttagaagagattattatttatggggtgtagaagaagattctcaacagtctactgtaaatcaccgcagtactgacactcgctcaactgttgaccatagcagcagtaccaatagttctttagacgggaacgaagaaatggcccggtcggaaaagataacaccatctcatcaagaaaagcctagcgataggtga

SEQ ID NO: 2 - protein, 491 aa, Saccharomyces sp.
MESRSTGPLTTEIYDGPTVAFMILGAALVFFMVPGLGFLYSGLARRKSALALIWVVLMATLVGILQWYFWGYSLAFSKSATNNKFIGNLDSFGFRNVYGKKSDGDVYPELAYATFQMMFSCVNLSIIAGATAERGRLLPHMVFLFILATIVYCPVTYWIWSPGGWAYQWGVLDWAGGGNIEILSAVSGFVYSWFLGKRNEKLLINFRPHNVSLVTLGTSILWFGWLLFNSASSLSPNLRSVYAFMNTCLSAITGGMTWCLLDYRSERKWSTVGLCSGIISGLVAATPSSGCITLYGSLIQGIVAGIVCNFATKLKYYAKVDDAMDILAEHGVAGIIGLIFNALFAADWVIGMDGVSEHEGGWISHNYKQMYKQIAYIAASIGYTAVVTAIICFVLGYIPGMTLRISDEAEERGMDEDQIGEFAYDYVEVRRDYYLWGVEEDSQQSTVNHRSTDTRSTVDHSSSTNSSLDGNEEMARSEKITPSHQEKPSDR

SEQ ID NO: 3 - DNA, 1479 bp, Saccharomyces sp.
atggagagtcgaactacagggcctttaacgactgaaacctacgatggccccactgtggccttcatgatattaggtgccgccctagtattttttatggtgcccggatttggattcttgtactccggattggcaagaaggaagtctgcactagcactaatctgggttgtattaatggcgactttggtcggtatactgcaatggtatttctggggttactctctagctttttcaaagtccgctccgaataataaattcattgggaatctagattcgtttggctttagaaacgtgtacggaaaaaaattcgatgaagatgcctaccctgagctcgcgtatgcaaccttccaaatgatgttttcgtgcgtcaacttaagtattatcgctggcgccactgccgaaagaggcaggctgctaccgcacatggtttttctctttattctagctaccattggatattgtccagtgacgtattggatttggtcaccaggtggttgggcataccaatggggagtcctcgattgggcaggcggcggcaacattgaaatattaagcgctgtttccgggtttgtttactcttggtttttgggcaaaagaaatgaaaagttactgataaatttcaggcctcataatgtttcattggtcactctaggcacatccatactgtggtttggctggctgctatttaattctgcatcctcattatccccaaatttgaggtcagtttatgcattcatgaatacatgtctcagtgccattactggtgggatgacgtggtgtcttctggattacagatcggagaagaaatggtcgacagttggtctgtgctccggtatcatttctgggctggtggctgcaacgccaagctcaggctgtataaccctttacggttcacttattcaaggcattgtggcgggggtagtgtgtaactttgcgacgaagttgaaatactacgctaaagtagatgatgccatggacattctagctgagcacggggttgcaggcgtaataggactaattttcaatgccctttttggagcagactgggtcattggtatggatggcactacagagcacgagggcggctgggtaactcacaattacaagcaaatgtataagcagatcgcttacattgccgcatccattgggtacactgctgctgtaactgcaataatctgctttgtgctcggctacatacccggtatgaggctaagaatatcagaagaggcagaggaggcgggtatggacgaagatcaaattggcgaatttgcgtacgattatgtggaagtgagaagagattactatctatggggtgtagacgaagattcacaacgctctgatgtaaatcaccgggtgaacaacgctcatttggccgctgaacgtagcagtagcggtactaatagttcctcggatgggaatggagaaatgattcaatccgaaaagatcctaccaattcatcaagaagatcctgccaataggtaa

SEQ ID NO: 4 - protein, 492 aa, Saccharomyces sp.
MESRTTGPLTTETYDGPTVAFMILGAALVFFMVPGFGFLYSGLARRKSALALIWVVLMATLVGILQWYFWGYSLAFSKSAPNNKFIGNLDSFGFRNVYGKKFDEDAYPELAYATFQMMFSCVNLSIIAGATAERGRLLPHMVFLFILATIGYCPVTYWIWSPGGWAYQWGVLDWAGGGNIEILSAVSGFVYSWFLGKRNEKLLINFRPHNVSLVTLGTSILWFGWLLFNSASSLSPNLRSVYAFMNTCLSAITGGMTWCLLDYRSEKKWSTVGLCSGIISGLVAATPSSGCITLYGSLIQGIVAGVVCNFATKLKYYAKVDDAMDILAEHGVAGVIGLIFNALFGADWVIGMDGTTEHEGGWVTHNYKQMYKQIAYIAASIGYTAAVTAIICFVLGYIPGMRLRISEEAEEAGMDEDQIGEFAYDYVEVRRDYYLWGVDEDSQRSDVNHRVNNAHLAAERSSSGTNSSSDGNGEMIQSEKILPIHQEDPANR

SEQ ID NO: 5 - DNA, 40 bp, Artificial
  Primer
gagctcatagcggccatggaaacgatggaaagtcgatcta

SEQ ID NO: 6 - DNA, 42 bp, Artificial
  Primer
ggatcctatgcggccgcgtataaagcaaatgcagatctatac

SEQ ID NO: 7 - DNA, 40 bp, Artificial
  Primer
gagctcatagcggccatggagagtcgaactacagggcctt

SEQ ID NO: 8 - DNA, 42 bp, Artificial
  Primer
ggatcctatgcggccgcatgcacatctatacatatatgaagg